(19) United States
(12) Patent Application Publication
(10) Pub. No.: US 2007/0265620 A1
(43) Pub. Date: Nov. 15, 2007

(54) VASCULAR COAGULATION FORCEPS

(75) Inventors: Mathias Kraas, Haseldorf (DE); Andreas Muckner, Hamburg (DE); Stephan Klockner, Hamburg (DE)

Correspondence Address: RANKIN, HILL, PORTER & CLARK LLP, 38210 Glenn Avenue, WILLOUGHBY, OH, 44094-7808, US

(73) Assignee: OLYMPUS WINTER & IBE GMBH, Hamburg (DE)

(21) Appl. No.: 11/746,879

(22) Filed: May 10, 2007

(30) Foreign Application Priority Data
May 15, 2006 (DE) 102006022913.4

(51) Int. Cl. A61B 18/14

(52) U.S. Cl. 606/51

(57) ABSTRACT

A surgical, vascular coagulation forceps (1) comprising two mutually displaceable jaws (3, 4) bearing flat electrodes (12, 13) connected to different terminals of an hf source (16), or one of said jaws being fitted with an ultrasonic oscillator, characterized in that a coil unit (18) connected to an eddy current sensor (20) is mounted on at least one of the jaws (3, 4).

### BACKGROUND OF THE INVENTION

The present invention relates to a vascular coagulation forceps or clamp including two mutually displaceable jaws bearing flat electrodes.

Such forceps are used in surgery to grip between their jaws blood vessels to be severed to squeeze them into a closed position and to heat them in said position by applying ultrasonics to or an hf current through them, the tissue coagulating thereby and hence sealing the blood vessel. Thereupon, using a cutter on the forceps or separate scissors, the blood vessel can be severed at its coagulated site.

High requirements apply to such coagulation. The thermal tissue coagulation must have advanced enough to reliably seal the blood vessel, for instance, and also to reliably seal the blood vessel which, for instance, also should withstand high arterial pressure. Accordingly, reliable bonding must be attained while ensuring that tissue strength be retained even if its coagulation should be excessive.

Conventionally, and taking into account the blood vessel size, the coagulation duration is controlled using empirical values, i.e. tables and the like. This method however entails uncertainties.

The objective of the present invention is to increase coagulation reliability of forceps of the above species.

### BRIEF SUMMARY OF THE INVENTION

In the present invention, the forceps is fitted with a coil unit allowing the measuring of eddy currents in the zone of coagulation.

Eddy currents are known in a variety of fields. They may also be used for body tissue measurements. The eddy currents are generated by an alternating current (AC) passing through the coil and they are circular currents induced at the measurement site situated on the coil axis. The circular induced currents may be measured using a separate testing coil or by their effect on generator coil current. Eddy current measurement may be carried out, for instance, at different frequencies and foremost provide inferences regarding the test site's electrical conductivity. As regards body tissue, said conductivity depends foremost on the tissue's liquid content.

In the present case of monitoring tissue coagulation wherein the tissue liquid content decreases with coagulation, measuring the eddy current is well suited to monitor the progress of coagulation. The eddy current measuring means, hereafter eddy current sensor, thereby allows ascertaining the time at which coagulation is optimal and shall be terminated.

The coil unit of the present invention may consist of one or several coils such as a generator coil and a measuring coil, further it may comprise additional coils for instance used for compensation.

To increase accuracy of measurement, the coil axis should run through the zone of coagulation. This goal is attained by the the coil unit being configured parallel to the gripping surface of a jaw. The coil unit is configured inside the active surface of a forceps jaw, thereby meeting the requirement its axis passes through the zone of coagulation.

The features of a forceps wherein the coil unit encloses the electrode advantageously relate to a bipolar, hf-loaded forceps. This offers a very compact coil configuration geometry for the measurements of the present invention.

The present invention is shown in illustrative and schematic manner in the appended drawing.

### BRIEF DESCRIPTION OF THE DRAWINGS

FIG. 1 is a sideview of a bipolar forceps fitted with an eddy current sensor, and

FIG. 2 is a section along line 2-2 of FIG. 1.

### DETAILED DESCRIPTION OF THE INVENTION

In a much simplified embodiment mode, the Figures show a bipolar forceps 1 with two jaws 3, 4 which extend proximally into handles 5, 6 and are articulating with each other by a pivot pin 7. The forceps also may be designed with another kind of pivot, to form a laparoscopic stem forceps while retaining essentially the same jaws 3, 4.

The two forceps elements 3, 5 and 4, 6 shown in the present embodiment are entirely metallic. Electrically insulating plates 10, 11 rest on the engaging surfaces 8, 9 that are displaced toward each other when the forceps 1 is closing and in turn support metal electrode plates 12, 13 connected by electrical conductors 14, 15, installed at the jaws 3,4 and the handles 5, 6, to the two terminals of a high frequency source 16.

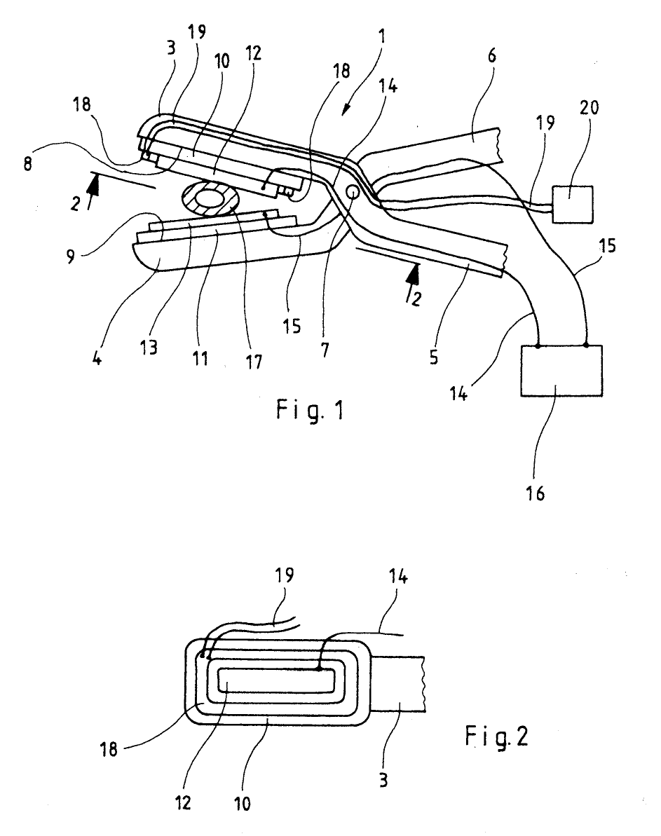

As shown in FIG. 1, the bipolar forceps 1 is used to seal a blood vessel, for instance an artery 17 shown sectionally in FIG. 1. As further shown in FIG. 1, the artery is seized between and squeezed by the jaws 3, 4 of the forceps 1 until its lumen has been closed. Thereupon and while maintaining the sealing compression, the hf source is turned ON and a current passes between the electrode plates 12 and 13 through the tissue of the artery 17. This current heats and coagulates the said tissue.

After a given time, coagulation has progressed enough that the superposed artery surfaces have fused into each other and the artery henceforth shall stay closed even in the presence of substantial internal pressure. At this time the hf current may be turned OFF and the forceps 1 may be released. Using separate scissors or an omitted cutting element on the bipolar forceps 1, the artery now can be severed at the center of the zone of coagulation.

The progressing coagulation is monitored at the shown bipolar forceps 1 in order to attain optimal coagulation and optimal mechanical tissue strength.

For that purpose a coil 18, which rectangularly encloses the electrode plate 12 and rests on the electrically insulating plate 10, is mounted on the jaw 3 and is connected by a two-conductor cable 19 to an eddy current sensor 20.

The eddy current sensor 20 transmits through the cable 19 an AC current into the coil 18. Said coil generates an AC magnetic field which in turn generates eddy currents about said coil's axis that is perpendicular to the gripping surface 8, said eddy currents being situated in the zone of coagulation, that is in the tissue of the artery 17. These eddy currents influence the current in the coil 18. This phenomenon can be measured by appropriate elements in the eddy current sensor 20. The inductive reaction foremost depends on the electrical conductivity of the arterial tissue, which in turn depends foremost on the tissue's water content. Said water content drops as coagulation progresses, whereby the eddy current sensor 20 is able to monitor said progress and display it (omitted).

In the embodiment mode shown, a coil 18 is connected by a two-conductor cable 19 to the eddy current sensor. A double-coil unit also may be used, wherein the coil 18 consists of two superposed coil parts each connected by a two-conductor cable to the eddy current sensor 20. One of these coil elements may be a generator coil and the other the sensing coil. Sensitivity of measurement may be increased in this manner. The coil unit 18 moreover may contain still other coil elements used for compensation.

In the shown embodiment mode, the tissue of the artery 17 is heated by an hf current. In another, omitted embodiment mode, the forceps also may heat said tissue ultrasonically. For that purpose one of the electrode surfaces 12, 13 may be designed as the surface of an ultrasonic oscillator which is fed from an ultrasonic generator at one of the handles 5, 6 for instance by means of an ultrasonic guide. The eddy current sensor may be designed in this instance in the manner discussed in relation to the shown bipolar forceps.

1. A surgical, vascular coagulation forceps (1) comprising:
two mutually displaceable jaws (3, 4) bearing flat electrodes (12, 13);
said flat electrodes (12, 13) being connected to different terminals of an hf source (16), or one of said jaws being fitted with an ultrasonic oscillator, and
wherein a coil unit (18) connected to an eddy current sensor (20) is mounted on at least one of the jaws (3, 4).

2. Forceps as claimed in claim 1, wherein the coil unit (18) is configured parallel to the gripping surface (8) of a jaw (3).

3. Forceps as claimed in claim 2, wherein the coil unit (18) encloses the electrode (12).
